(19) United States
(12) Patent Application Publication
(10) Pub. No.: US 2024/0140872 A1
(43) Pub. Date: May 2, 2024

(54) GYPSUM COMPOSITION FOR MOLD PRODUCTION

(71) Applicant: YOSHINO GYPSUM CO., LTD., Tokyo (JP)

(72) Inventors: Rina KOHARA, Tokyo (JP); Tatsuya HORIUCHI, Tokyo (JP); Emi KAMEDA, Tokyo (JP); Narihiro KUMAGAI, Tokyo (JP)

(21) Appl. No.: 18/547,782

(22) PCT Filed: May 11, 2022
(86) PCT No.: PCT/JP2022/019938

§ 371 Date: Aug. 24, 2023

(30) Foreign Application Priority Data
Jun. 2, 2021 (JP) 2021-093091

(51) Int. Cl. C04B 28/14; C04B 40/00

(52) U.S. Cl. CPC C04B 28/147; C04B 40/0032; A61C 13/20

(57) ABSTRACT

An object of the present invention is to provide a mold-making gypsum composition that can contribute, in a case of, for example, making a model of dentures, to improvements in workability in a step of making a gypsum mold (mold) which is necessary for making a model of artificial gingiva (gum) part made of a resin and which is different individually and is required to have an elaborate shape. In detail, the present invention provides a mold-making gypsum composition such that a slurry-like malaxated product obtained by adding water to the mold-making gypsum composition exhibits favorable fluidity and therefore the mold-making gypsum composition is used for tertiary investing for filling a void in a polymerization flask in making a mold, and thereby workability can be improved and a favorable mold in which occurrence of cracks of gypsum is suppressed when the mold-making gypsum composition is set can be formed. The present invention is a cast-making gypsum composition containing gypsum hemihydrate, wherein the gypsum hemihydrate contains α gypsum hemihydrate and β gypsum hemihydrate in a ratio of 25:75 to 65:35.

### TECHNICAL FIELD

The present invention relates to a gypsum composition for making a mold made of gypsum to be used in making a model and relates to, for example, a mold-making gypsum composition suitable for making a model of dentures (false teeth). In more detail, the present invention relates to a mold-making gypsum composition such that a slurry-like malaxated product obtained by adding water to the mold-making gypsum composition exhibits favorable fluidity and therefore workability in making a mold (gypsum mold) can be improved and a favorable mold in which occurrence of cracks of gypsum is suppressed when the mold-making gypsum composition is set can be formed.

### BACKGROUND ART

Gypsum compositions have been used as gypsum molds (molds) in making various models and have properties of faithfully reversing fine patterns of the models to make it possible to give an extremely smooth surface. An example of the models for which such properties are required is so-called dentures. In response to society with longer life expectancy, better-fitting full dentures and partial dentures are in demand, and a variety of materials (see, Patent Literature 1) to be used for artificial teeth and artificial gingiva (gum) and apparatuses for making artificial teeth and artificial gingiva (gum) have been developed. Under such circumstances, a gypsum composition that is a useful material in making a mold which is necessary for making a model, such as a model of dentures, which has a different shape for every model and which is also required to have elaborateness, is required to have properties of making it possible to make a favorable mold, so-called gypsum model, with better workability and more stably. Hereinafter, a mold obtained using a gypsum composition is also referred to as a gypsum mold.

Hereinafter, description will be made using making “dentures” as one example of the models having the above-described properties. According to studies conducted by the present inventors, an “issue of an improvement in workability in a stage of forming a mold” which is necessary in making artificial gingiva (gum) to be formed with a resin depends on the properties of a gypsum composition to be used for forming a mold. That is, the workability and setting time for a gypsum composition, and the like in filling the gypsum composition give a significant influence on the efficiency of mold formation operation. In addition, the surface state of the gingiva (gum) part formed by pouring a resin in the cavities (spaces) of a resultant mold and setting the resin depends on the inner surface state of the cavities of a gypsum mold to be used. To make better-fitting “dentures,” the following are required as the basic performance. That is, desired are: when dentures are placed in the mouth, artificial teeth are fixed and held to artificial gingiva (gum) in such a way as to make good occlusion of teeth; and further, the surface state of the artificial gingiva (gum) part formed with a resin does not give a feeling that something is wrong to a user who wears the dentures. Therefore, in the production of “dentures,” a favorable mold that can form artificial gingiva (gum) made of a resin, which is different individually and has an elaborate shape, and a resin material for forming favorable artificial gingiva (gum) using the mold are required.

As described above, as well as the artificial teeth, the perfection of the artificial gingiva (gum) part made of a resin, which supports and integrates the artificial teeth, is also an important factor, and therefore studies on a gypsum composition to be used for making the artificial gingiva (gum) part made of a resin are desired to be conducted. Patent Literature 1 has proposed a resin material for dentures which are formed using the mold. In addition, it is important for a producer of dentures to supply dentures having excellent compatibility as soon as possible. With regard to this, working efficiency during making dentures (or a model of dentures) by a dental technician is particularly desired to be improved, and when the working efficiency can be improved, it is also worthwhile to users of the dentures.

### CITATION LIST

### Patent Literature

Patent Literature 1: Japanese Patent Laid-Open No. 2020-204011

### SUMMARY OF INVENTION

### Technical Problem

In a series of complicated steps of making dentures (or a model of dentures), an examples of the steps that impair the workability of a dental technician is the following step. That is, the workability is impaired in the “step of making a mold (gypsum mold) for forming artificial gingiva (gum) part made of a resin” which is necessary in making final “dentures” in such a way that the resin is poured into the cavity of the mold to form the artificial gingiva (gum) part made of the resin and then artificial teeth and the artificial gingiva (gum) made of the resin are integrally formed. According to studies conducted by the present inventors, as will be described, the workability of a dental technician is particularly impaired at the stage of the above-described step of making a mold which is different individually and which is required to have an elaborate shape in order to reproduce the shape of the gums, and when this can be dissolved by improving the mold-making gypsum composition, it is extremely useful.

Accordingly, an object of the present invention is to provide a mold-making gypsum composition that can contribute to improvements in workability in the step of making a mold which is necessary in making, for example, an artificial gingiva (gum) part made of a resin and which is different individually and is required to have an elaborate shape.

### Solution to Problem

The above-described object has been achieved by the present invention described below. That is, the present invention provides a mold-making gypsum composition described below.
[1] A mold-making gypsum composition comprising gypsum hemihydrate, wherein the gypsum hemihydrate comprises α gypsum hemihydrate and β gypsum hemihydrate in a ratio of 25:75 to 65:35.

Preferred embodiments of the mold-making gypsum composition of the present invention include the following invention.
[2] The mold-making gypsum composition according to [1], wherein the ratio of α gypsum hemihydrate to β gypsum hemihydrate is 25:75 to 60:40.
[3] The mold-making gypsum composition according to [1], wherein the ratio of α gypsum hemihydrate to β gypsum hemihydrate is 30:70 to 60:40.
[4] The mold-making gypsum composition according to any one of [1] to [3], wherein the gypsum hemihydrate accounts for 95 parts by mass or more of 100 parts by mass of the mold-making gypsum composition.
[5] The mold-making gypsum composition according to any one of [1] to [4], further comprising a water-reducing agent, wherein the amount of the water-reducing agent added is 0.02 to 0.2 parts by mass based on 100 parts by mass of the gypsum hemihydrate.
[6] The mold-making gypsum composition according to any one of [1] to [5], wherein a pot life of a slurry-like malaxated product obtained by adding water to the mold-making gypsum composition in such a way that the mixing water amount is 45% and malaxating the water and the mold-making gypsum composition is adjusted to be 8 minutes or longer, and a setting time thereof is adjusted to 30 minutes or shorter.

### Advantageous Effects of Invention

The present invention makes it possible to provide a useful mold-making gypsum composition (hereinafter, referred to as the gypsum composition for a mold) that can effectively contribute to improvements in workability in a step of making a mold which is necessary in making a model and which is, as exemplified above, different individually and is required to have an elaborate shape.

### BRIEF DESCRIPTION OF DRAWINGS

FIG. 1 is a schematic cross-section view for describing the situation in which a gypsum composition for a mold of the present invention is used, the schematic cross-section view illustrating the inside of a polymerization flask 10. In the polymerization flask, “wax dentures 2 having artificial teeth 2b and a wax occlusion rim 2a” supported by a gypsum model (hereinafter, also referred to as “working model”) 3 of a patient's oral cavity, prepared by pouring gypsum in impression of a patient, taken in a dentist's office or the like, need to be embedded without a gap in the gypsum composition.

FIG. 2A is a schematic oblique view for describing a state in which the “wax dentures 2 having artificial teeth and a wax occlusion rim” have been disposed on a gypsum composition 4 for primary investing, filled in a container 5a which is a lower-side constituent member of the polymerization flask, in a step of making a mold to be used for making artificial gingiva made of a resin.

FIG. 2B is a schematic oblique view for describing a state of performing an operation of embedding the “wax dentures 2 having artificial teeth and a wax occlusion rim” shown in FIG. 2A and disposed in the lower-side container 5a that constitutes the polymerization flask with a gypsum composition 6 for secondary investing.

FIG. 3 is a schematic oblique view of an example of the polymerization flask to be used in the step of making a mold, and the inside of the polymerization flask needs to be made into a state as shown in FIG. 1 in the step of making a mold. That is, the inside of the polymerization flask 10 needs to be filled with the gypsum composition in such a way as to have no void when an upper-side member 5b to be a lid is stacked on the lower-side container 5a of the polymerization flask.

FIG. 4 is a schematic oblique view for describing how a slurry-like malaxated product 1, obtained by adding water to a gypsum composition for a mold of the present invention, is poured into a gypsum-composition-unfilled void formed in the polymerization flask which is in the state shown in FIG. 3.

### DESCRIPTION OF EMBODIMENTS

Hereinafter, the present invention will be described in more detail giving preferred embodiments. First of all, a step of making a mold which is necessary in making artificial gingiva will be described, wherein the step, which the present inventors regard as a problem, is a cause for impairing the workability of a dental technician in a series of complicated steps of making dentures. Description is made below on making full dentures (hereinafter, referred to as “dentures”) as a representative example. In a series of complicated steps of making the “dentures”, the outline of a step of making artificial gingiva (gum) made of a resin, in which artificial teeth are fixed and supported, is as follows.

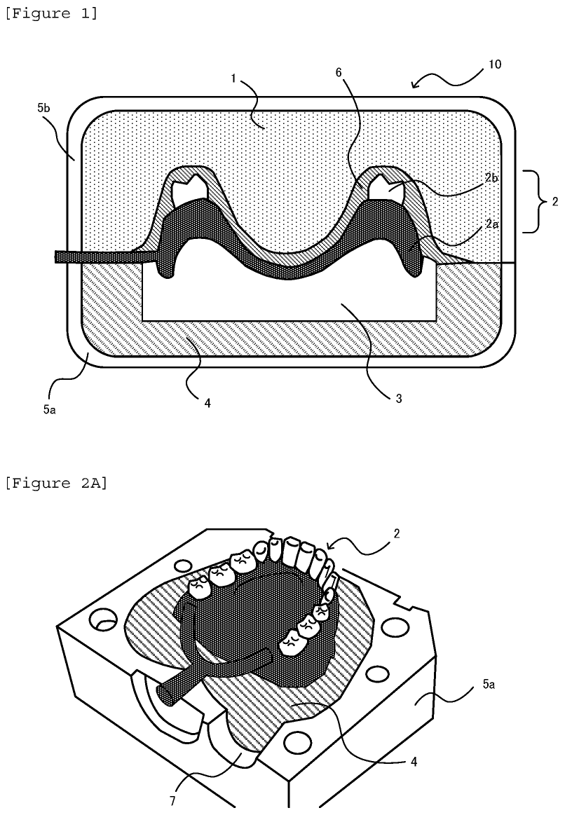

Firstly, the “wax dentures having artificial teeth and a wax occlusion rim” are made in the manner as described below, and next, a mold made of gypsum for making the artificial gingiva (gum) made of a resin is made using the wax dentures. The gypsum composition for a mold of the present invention is used at a part which occurs in a polymerization flask in making this mold and is not filled with gypsum, resulting in effective contribution to enhancement of the workability in the step of making the “dentures.” Described below is an example of using a polymerization flask 10 having a structure that is separable into an upper part and a lower part as shown in FIGS. 1 to 3. That is, as shown in the Figures, a polymerization flask composed of a lower-side member 5a, which is a container, and an upper-side member 5b (sometimes also referred to lid 5b or lid), which is a lid, is used.

Firstly, a wax occlusion rim 2a is provided on a working model 3, and artificial teeth 2b are disposed at a position of the wax occlusion rim where favorable occlusion to a user of the dentures can be realized (see, FIG. 1). The wax occlusion rim is formed with a material, such as wax, which is melted with a microwave oven or boiling water. Together with this operation, the surface part of the wax occlusion rim 2a which is a support for the artificial teeth 2b is engraved with the artificial teeth 2b being fixed to the wax occlusion rim 2a to reproduce the gingiva (gum) part visually well. In this manner, the “wax dentures 2 having artificial teeth 2b and a wax occlusion rim 2a” is made.

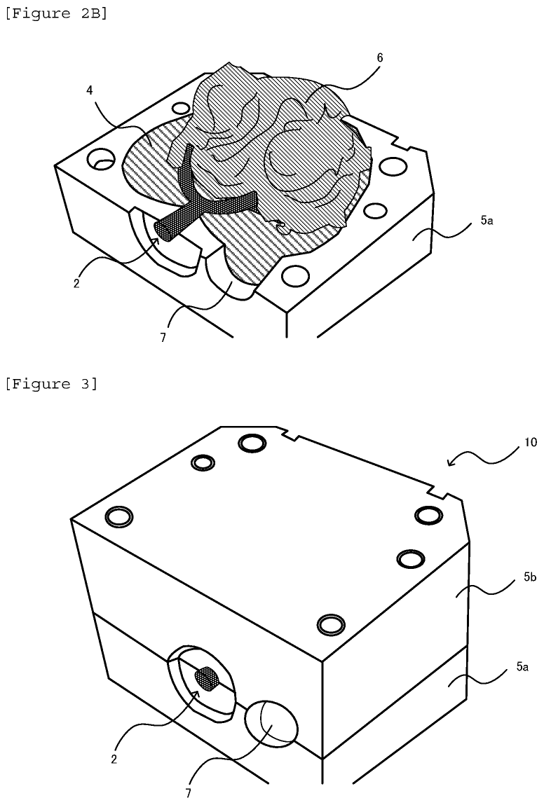

Subsequently, as shown in FIG. 2A, the “wax dentures 2 having artificial teeth and a wax occlusion rim” obtained in the manner as described above are fixed, using the gypsum composition 4 for primary investing, inside the above-described lower-side container 5a of the polymerization flask 10 having a structure that is separable into an upper part and a lower part. This operation is referred to as “primary investing.”

Subsequently, as shown in FIG. 2B, the gypsum composition 6 for secondary investing is disposed on the “wax dentures 2 having artificial teeth and a wax occlusion rim” after primary investing, obtained in the manner as described above, to cover the wax occlusion rim 2a and the artificial teeth 2b. This operation is referred to as “secondary investing.”

Subsequently, the upper-side lid 5b is put on the polymerization flask, and then a slurry-like malaxated product composed of the gypsum composition for a mold of the present invention and water is poured into the polymerization flask 10 to fill the void which has occurred inside the polymerization flask 10 to embed the “wax dentures 2 having artificial teeth and a wax occlusion rim” in the gypsum composition. This operation is referred to as “tertiary investing.” Hereinafter, the “wax dentures 2 having artificial teeth 2b and a wax occlusion rim 2a” is sometimes simply referred to as “dentures 2.”

The above-described series of investing steps make it possible to obtain a mold for forming an artificial gingiva (gum) part made of a resin, the mold being one in which the artificial teeth 2b are supported and fixed with the occlusion being adjusted. In the present invention, by utilizing, as described above, the gypsum composition for a mold of the present invention wherein a slurry-like malaxated product obtained by adding water to the gypsum composition exhibits favorable fluidity in the operation of tertiary investing in obtaining this mold for forming artificial gingiva, obtaining with good workability a favorable artificial gingiva (gum) made of a resin can be realized, as will be described later. The procedure of making the artificial gingiva (gum) part of the wax dentures 2 with a resin using the mold (gypsum mold) for forming artificial gingiva is as follows.

Firstly, gypsum containing the slurry-like malaxated product used for tertiary investing, the gypsum filled in the polymerization flask, is sufficiently set, and then the wax (wax occlusion rim 2a) forming the artificial gingiva (gum) part of the wax dentures 2 is sufficiently melted out using a microwave oven or boiling water to form a mold (gypsum mold) in which the wax part has been melted and become a cavity. The resultant mold is one in which the artificial teeth 2b, which were held at predetermined positions by the wax before being melted, have been held at the predetermined positions by the set gypsum. In addition, in the inner surface of the cavity after the wax is removed, formed by the wax having melted and flowed out, the state of the engraved gingiva (gum) has been transferred.

Subsequently, the resin for forming the artificial gingiva is filled in this cavity of the gypsum mold to set the resin. Finally, the mold is broken to take out the artificial gingiva (gum) made of the resin and the artificial teeth integrated with the artificial gingiva by being strongly fixed and supported by the artificial gingiva, and then the artificial teeth and the artificial gingiva part are carefully polished. This makes it possible to obtain the “dentures” in which the artificial teeth are favorably disposed with the occlusion as adjusted on the artificial gingiva (gum) made of the resin.

The present inventors have conducted diligent studies on the improvement in workability in the series of the above-described steps and, as a result, found that the improvement in workability can be realized by improving a gypsum composition for a mold for utilization in the operation which has a problem as described below. In the step of filling a resin in the mold in which the wax part has been melted to form a cavity, the resin is usually injected, for the purpose of densely filling the resin, by pressurization into the cavity formed after the wax is removed. Therefore, there is a risk that the smoothness of the surface of the formed gingiva (gum) made of the resin is impaired because cracks or breakage occur in the set product of the gypsum composition used for the secondary investing and the parts where the cracks or breakage have occurred become burrs. In addition, from the reason that a conventional gypsum composition which has been used for tertiary investing is such that a malaxated product thereof with water has poor fluidity, and in addition, from the requirement of exhibiting the strength of the set gypsum composition, the gypsum composition for tertiary investing generally needs to be filled carefully by applying vibration with a vibrator so that a void will not occur in the polymerization flask.

Under the circumstances as described above, when the gypsum composition for a mold of the present invention is used for the tertiary investing, the “wax dentures 2 having artificial teeth 2b and a wax occlusion rim 2a” are quickly embedded in the gypsum composition 6 used for the secondary investing and the gypsum composition 1 which has been poured in the form of slurry prepared using the gypsum composition for a mold of the present invention and which has been used for the tertiary investing, as shown in FIG. 1 and FIG. 2A. According to studies conducted by the present inventors, as a result of this, a set product of the gypsum composition for a mold of the present invention, used for tertiary investing, gives a surprising effect of exhibiting strength sufficient for suppressing cracks and breakage caused by pressure during injecting the resin together with a set product of the gypsum composition used for the secondary investing.

The polymerization flask 10 which is used for the above-described steps can be of various shapes, and examples of the polymerization flask 10 includes not only one that is separable into two parts, exemplified above, but also one having a structure that is separable into three parts. However, finally, as described above, set gypsum needs to be taken out of the polymerization flask filled with the gypsum and further the gypsum needs to be broken to take out the dentures, and therefore the polymerization flask, whatever the structure may be, has a structure that is separable into an upper part and a lower part, as schematically shown in FIG. 1 to FIG. 3. Then, as shown in FIG. 2A and FIG. 2B, the gypsum composition 6 for secondary investing, having a high viscosity, is disposed, from above the container 5a which is separated and open, on the “wax dentures 2 having artificial teeth and artificial gingiva (gum)” disposed in the lower-side container 5a which is a receptacle of the polymerization flask and fixed by the gypsum composition 4 for primary investing in such a way as to cover the “wax dentures 2 having artificial teeth 2b and a wax occlusion rim 2a.”

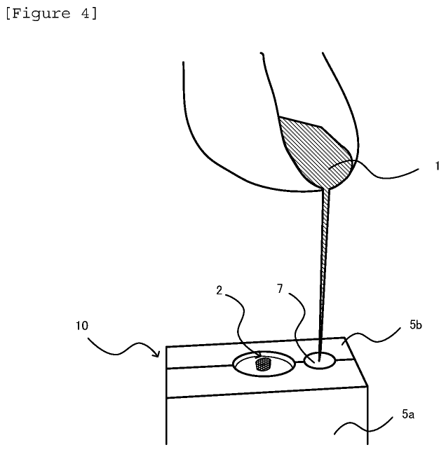

Thereafter, the upper-side member 5b that functions as a lid of the container 5a is stacked on the lower-side container 5a of the polymerization flask 10 to integrate the upper-side member 5b and the lower-side container 5a as shown in FIG. 3, and then the gypsum composition is set. When the upper-side member 5b which is a lid is stacked on the container 5a of the polymerization flask to integrate the upper-side member 5b and the container 5a after the secondary investing, an unfilled void part where the gypsum is not filled is formed in the polymerization flask 10 after the integration although the size of the void part may differ depending on the case. Therefore, a tertiary investing operation of filling the gypsum composition in this void part needs to be additionally performed. Specifically, for example, there has been performed a tertiary investing operation of filling the gypsum composition from a hole 7, as shown in FIG. 3, which is provided on the side surface of the polymerization flask 10 and which leads to the unfilled void part where the gypsum is not filled in the polymerization flask. On this occasion, in the conventional techniques, α gypsum composition having high viscosity, the gypsum composition prepared for tertiary investing, has been filled gradually and carefully over time using a vibrator or the like so that an unfilled void part will not occur, and thus the gypsum composition has been filled in the polymerization flask.

In the conventional techniques, the “wax dentures 2 having artificial teeth 2b and a wax occlusion rim 2a” are carefully embedded, in the manner as described above, over time in the gypsum composition so that a void will not occur, the gypsum is then set, and thereafter a gypsum set product, which is for making a mold and in which the wax dentures 2 have been embedded inside, is taken out of the polymerization flask 10. The present inventors have become aware that the cause for impairing the workability of a dental technician in the above-described step of making a mold for dentures is a step (tertiary investing) of filling the gypsum composition in the “void which has occurred in the polymerization flask and which is not filled with the gypsum”, which is inevitable due to the structure of the polymerization flask.

When a gypsum composition having the following properties can be developed to address the above-described technical problems, it is extremely useful in terms of work. Specifically, development of a gypsum composition for mold production, having the following properties, is desired. That is, the gypsum composition can be filled, as tertiary investing, with a favorable state quickly and easily in the void which has occurred in the polymerization flask; a slurry-like malaxated product has excellent fluidity; and in addition, burrs do not occur at the resin part which is formed using a mold (gypsum mold) having a cavity formed after the wax occlusion rim 2a is removed and which is to be artificial gingiva (gum).

Facing the above-described technical problems in making a mold in the step of making the dentures, the present inventors have conducted diligent studies to develop a gypsum composition capable of exhibiting the above-described properties to reach the present invention. Specifically, the gypsum composition for mold production of the present invention contains gypsum hemihydrate, wherein the gypsum hemihydrate contains α gypsum hemihydrate and β gypsum hemihydrate in a ratio of 25:75 to 65:35.

When water is added to the gypsum composition for a mold, having a composition as described above, to make the gypsum composition into a slurry form and then perform tertiary investing of filling the gypsum composition in a void which has occurred in the polymerization flask and which is not filled with the gypsum, the gypsum composition can be filled in the void inside the polymerization flask densely, quickly, and easily because of having adequate fluidity. Therefore, the working efficiency in making a mold (gypsum mold) for making artificial gingiva (gum) made of a resin is outstandingly improved.

Further, by filling, in the manner as described above, the gypsum composition for a mold of the present invention in the polymerization flask by the tertiary investing, the excellent effects, which will be described later, are obtained. As described above, in the step of making a mold (gypsum mold) for making artificial gingiva (gum), the following operations are performed. After the gypsum compositions for the primary, secondary, and tertiary investing are filled in the polymerization flask, the gypsum is sufficiently set, wax (wax occlusion rim) is melted out to form a cavity and complete a mold (gypsum mold), and the resin to be artificial gingiva (gum) is filled in the cavity. Thereafter, the polymerization flask is disintegrated to take out the mold from inside the flask, and the mold is broken to take out a model (dentures) from the mold. Here, the mold which is obtained in the manner as described above and includes the model (dentures) is integrated, without a problem, with the gypsum composition for a mold of the present invention, which is used for the tertiary investing, and the gypsum composition having high viscosity and being filled in advance in the polymerization flask by the secondary investing, which is performed prior to the tertiary investing and is performed for embedding the wax dentures. Therefore, when the resin to be the artificial gingiva (gum) is filled in the cavity of the mold, the gypsum composition for a mold of the present invention, used for the tertiary investing, and the gypsum composition used for the secondary investing work together to make the cracks and the like due to filling of the resin to be the artificial gingiva (gum) unrecognizable. Then, the artificial gingiva (gum) part which is taken out by breaking the mold, which forms the “dentures,” and which is made of the resin is in a favorable state in which burrs and the like which occur due to the cracks are not present. Therefore, the necessity for a post-treatment such as removing burrs can be reduced. In addition, the mold after setting the resin can be broken without a problem. Then, by carefully removing the gypsum adhering to the artificial teeth and the like, the “dentures” in a favorable state can finally be obtained.

Hereinafter, materials for forming the gypsum composition for a mold of the present invention, which provides the above-described excellent effects, will be described. First of all, the gypsum composition for a mold of the present invention is used for the applications as exemplified above and is not used for producing a mold (gypsum mold) for casting a metal. Therefore, refractoriness and high heat resistance are not required for the gypsum composition for a mold of the present invention, and accordingly, the gypsum composition for a mold of the present invention does not contain a refractory material, such as, for example, cristobalite or quartz. In addition, description is made herein taking the “dentures” as a model that needs a mold as an example, but the applications are not limited to the “dentures.” Besides, the gypsum composition for a mold of the present invention can widely be utilized in steps of making molds to be used in making models and the like made of a resin for various purposes. By using the gypsum composition for a mold of the present invention, various models of elaborate shapes in which the occurrence of burrs is reduced can efficiently be obtained.

In addition, the gypsum composition for a mold of the present invention is provided as a powder and is made into a slurry-like malaxated product by adding water when used. The “powder” as referred to in the present invention means a powder having an average particle size of 200 μm or smaller. The average particle size herein means a particle size in terms of median size (d₅₀) in particle size distribution measurement, determined by a laser diffraction/scattering method. Note that the average particle size of the gypsum composition for a mold in the present invention can be measured using a laser diffraction particle size distribution analyzer (trade name: Microtrac HRA manufactured by NIKKIAO CO., LTD.).

[Gypsum Hemihydrate]

The gypsum composition for a mold of the present invention contains gypsum hemihydrate. Specifically, a ½ hydrate of calcium sulfate (CaSO₄·½H₂O) is referred to as gypsum hemihydrate. Gypsum hemihydrate reacts with water to change into gypsum dihydrate, and therefore a gypsum slurry prepared by kneading gypsum hemihydrate and water is quickly set when injected into the polymerization flask. Therefore, gypsum hemihydrate is widely used for making a mold. Gypsum hemihydrate is obtained by calcining gypsum dihydrate (calcium sulfate dihydrate). Examples of the gypsum dihydrate which is a raw material to be used for the calcination include natural gypsum, by-product gypsum, and recycled gypsum obtained through steps of recycling gypsum products. Gypsum hemihydrate that constitutes the gypsum composition for a mold of the present invention may be one obtained from any of the above-described gypsum dihydrates which are raw materials. Here, the amount of gypsum hemihydrate in the gypsum composition for a mold of the present invention can be, for example, 95 parts by mass or more of 100 parts by mass the gypsum composition for a mold. The amount of gypsum hemihydrate can more preferably be 98 parts by mass or more.

The gypsum composition for a mold of the present invention contains the above-described gypsum hemihydrate and the composition of the gypsum hemihydrate is made in such a way as to contain α gypsum hemihydrate and β gypsum hemihydrate in a ratio of 25:75 to 65:35. Preferred embodiments of the gypsum composition for a mold of the present invention include the ratio of α gypsum hemihydrate to β gypsum hemihydrate being 25:75 to 60:40. More preferred embodiments include the ratio of α gypsum hemihydrate to β gypsum hemihydrate being 30:70 to 60:40. In addition, the average particle size of these gypsum hemihydrates is preferably about 10 to about 80 μm, more preferably about 20 to about 50 μm. Hereinafter, α gypsum hemihydrate and β gypsum hemihydrate, which are essential in the gypsum composition for a mold of the present invention, will be described. As will be described later, it is considered that by specifying the composition of gypsum hemihydrate as described above, the gypsum composition for a mold of the present invention provides the effects of the present invention, which have never been achieved so far.

(α Gypsum Hemihydrate)

α Gypsum hemihydrate can be obtained by subjecting gypsum dihydrate as described above as a raw material to pressure calcination in water or water vapor. α Gypsum hemihydrate can be malaxated in a smaller mixing water amount (that is, lower mixing water amount) than β gypsum hemihydrate which will be described later. Therefore, a gypsum set product obtained using α gypsum hemihydrate has high strength (compressive strength). As described above, α gypsum hemihydrate which is used in the present invention preferably has an average particle size of about 10 to about 80 μm, more preferably about 20 to about 50 μm.

(β Gypsum Hemihydrate)

β Gypsum hemihydrate is obtained by calcining gypsum dihydrate as described above as a raw material, in the air. The mixing water amount for malaxation needs to be made larger than that for α gypsum hemihydrate described above. Therefore, a gypsum set product obtained using β gypsum hemihydrate has low strength (compressive strength). According to studies conducted by the present inventors, when the mixing water amount for β gypsum hemihydrate is decreased in such a way as to be equal to the mixing water amount for α gypsum hemihydrate, a gypsum set product obtained using β gypsum hemihydrate has higher strength than a gypsum set product obtained using α gypsum hemihydrate. As described above, β gypsum hemihydrate which is used in the present invention preferably has an average particle size of about 10 to about 80 μm, more preferably about 20 to about 50 μm.

The main raw material which has conventionally been used for a mold-making gypsum composition to be used for making “dentures” is β gypsum hemihydrate. α Gypsum hemihydrate has not been used as the raw material is because of the following reason. To obtain dentures embedded in a mold, the dentures need to be taken out by breaking the mold (gypsum set product) finally. When α gypsum hemihydrate is used for a mold-making gypsum composition, the strength of the gypsum set product is too high, which makes it difficult to break the gypsum set product, and if the gypsum set product is forcibly broken, there is a concern that the dentures embedded in the mold are damaged. The dentures are individually made taking the occlusion of each user, appearance, and the like into consideration and are elaborate, and therefore the damage to dentures is not acceptable. Thus, even if α gypsum hemihydrate is used as a raw material for a mold-making gypsum composition, the amount has been relatively small. This is common general technical knowledge on gypsum compositions for molds which are used for molds (gypsum molds) for making “dentures” and the like from the above-described reason.

Under the current circumstances described above, the present inventors have recognized, as described above, that it is important to enable quick and easy filling of a gypsum composition in a void which occurs in a polymerization flask due to the structure and the like of the polymerization flask in order to improve the workability in making “dentures.” As a breakthrough of the above-described common general technical knowledge on gypsum compositions for molds, when it is possible to provide a gypsum composition that enables quick and easy filling of the gypsum composition in the void, which occurs in the polymerization flask and in which the gypsum composition is not filled, without using pressure injection or a special apparatus, such as a vibrator, or without requiring careful filling over long hours as in current circumstances even if a vibrator for example is used, it is extremely useful.

Under the above-described recognition, the present inventors have conducted diligent studies to find that a gypsum composition for a mold, which can achieve the above-described object, can be provided by extremely simple means of making the composition of gypsum hemihydrate raw materials that constitute a gypsum composition for a mold into one containing α gypsum hemihydrate and β gypsum hemihydrate in a ratio of 25:75 to 65:35 as specified in the present invention. That is, when α gypsum hemihydrate and β gypsum hemihydrate are used within the range of the ratio specified in the present invention, the fluidity of a gypsum composition can be made favorable by adding water to a powder material in a mixing water amount of, for example, 45% into a slurry form, thereby making it possible to realize quick and easy filling of the gypsum composition in a void (space) which occurs due to the structure and the like of the polymerization flask, which occurs in the polymerization flask, and in which the gypsum is not filled.

In addition, the slurry obtained using the gypsum composition for a mold of the present invention exhibits favorable fluidity, therefore makes a risk of mixing bubbles during kneading extremely low and makes a defoaming agent unnecessary. Alternatively, even if a deforming agent is added, the amount thereof can be outstandingly reduced. Thus, the gypsum composition for a mold of the present invention can provide another effect of being industrially useful in terms of costs for raw materials.

In addition to those described above, the malaxated product of the gypsum composition for a mold, having the above-described composition, of the present invention with water is useful one having the following properties even when produced through malaxation with a small amount of water. The malaxated product has low viscosity and excellent fluidity, and on top of that, in spite of the fact that the resultant gypsum set product is formed using a high ratio of β gypsum hemihydrate which makes the strength (compressive strength) low, the resultant gypsum set product has sufficient compressive strength such that a mold which is the gypsum set product obtained using the gypsum composition for a mold is never broken when a resin for forming artificial gingiva (gum) for dentures made of a resin is injected into the cavity of the mold. Further, after the resin is set, the mold can be broken without a problem and dentures (model) inside can be taking out by breaking the mold.

Further, the gypsum composition for a mold of the present invention is integrated favorably with the gypsum composition which is used in advance for the secondary investing and which has high viscosity, and thereby prevents cracks and the like from occurring in the set product of the gypsum composition used in the secondary investing. Therefore, the artificial gingiva (gum) made of a resin and obtained by supplying the resin for forming the gum part into the cavity of the obtained mold and setting the resin is made favorable without burrs. Thus, secondary processing after setting the resin is made easy, and therefore the gypsum composition for a mold of the present invention is superior in economic efficiency.

Furthermore, according to studies conducted by the present inventors, when the gypsum composition for a mold of the present invention contains a water-reducing agent (dispersant), the amount of the water-reducing agent added can be 0.02 to 0.2 parts by mass based on 100 parts by mass of gypsum hemihydrate having the above-described composition. That is, even when the amount of the water-reducing agent (dispersant) added is relatively small, as small as 0.2 parts by mass or less, the gypsum composition for a mold of the present invention can provide the effect of adding the water-reducing agent. Therefore, the gypsum composition of the present invention can also reduce costs for raw materials and is superior in economic efficiency from this point.

For example, a naphthalenesulfonic acid-based dispersant, a melamine-based dispersant, a polycarboxylic acid-based dispersant, or a phosphonic acid-based dispersant can be used as the water-reducing agent. Note that the water-reducing agent is not limited to these, and any of conventionally known water-reducing agents (dispersants) can be used.

The gypsum composition for a mold of the present invention may contain a conventionally known additive as long as it does not impair the expected object. Specifically, a setting modifier, such as a setting accelerator or a setting retarder, can appropriately be added. Examples of the setting accelerator include gypsum dihydrate and calcium sulfate, and other general setting accelerators can also be used. Examples of the setting retarder include sodium citrate, and other general setting retarders can also be used.

### EXAMPLES

Next, the present invention will more specifically be described giving Examples and Comparative Examples. Hereinafter, parts or % are on a mass basis unless otherwise noticed.

### Examples 1 to 5 and Comparative Examples 1 and 2

Gypsum compositions for a mold of Examples 1 to 5 and Comparative Examples 1 and 2 were prepared according to compositions shown in Table 1. In preparing each gypsum composition for a mold, 0.03 parts of gypsum dihydrate and 0.2 parts of calcium sulfate, as setting accelerators, 0.025 parts of sodium citrate, as a setting retarder, and 0.025 parts of potassium tartrate, as an expansion inhibitor, based on 100 parts by mass of the gypsum composition for a mold were added under the same conditions. Further, each composition was adjusted so as to have the following flow value for the purpose of making it possible to secure favorable fluidity in pouring the gypsum composition for a mold into the polymerization flask when the prepared composition was used for tertiary investing for making “dentures.” That is, a water-reducing agent in an amount shown in Table 1 was added to each gypsum composition in such a way as to make a flow value 115 mm to 135 mm in the case where: water was added to each powdery gypsum composition composed of the above-described combination; and a resultant mixture was malaxated to make a slurry-like malaxated product in which the mixing water amount was 45%.

[Evaluation Methods and Evaluation Results]

Evaluations were performed in the manners as described below using slurry-like malaxated products obtained by adding water to the powdery gypsum compositions for a mold of Examples and Comparative Examples, each having a composition shown in Table 1, in such a way that the mixing water amount was 45% and malaxating the resultant mixtures, or using gypsum set products obtained by setting the malaxated products. Evaluation results are shown in Table 1.

(Pot Life)

In dental rubber bowls, water (23±2° C.) was weighed so as to give predetermined mixing water amounts, and the powdery gypsum compositions for a mold of Examples and Comparative Examples, which are objects of the test, were put into the dental rubber bowls respectively over 10 seconds. Then, the resultant mixtures were left to stand still for 20 seconds and thereafter malaxated with a spatula for 60 seconds to obtain malaxated products. The stirring speed for the malaxation was set to 120 rpm in all the examples. Hereinafter, a slurry-like malaxated product obtained in the manner as described above refers to a “gypsum slurry.”

After the malaxation was completed, an operation of pouring a small amount of the gypsum slurry every 30 seconds was repeated. Then, the point in time 30 seconds before the viscosity of the gypsum slurry became high and the gypsum slurry no longer flowed continuously (the flow was cut in places while the gypsum slurry fell off) was defined as the time during which pouring is possible, so-called “pot life.” To secure the working time intended in the present invention, the pot life of 8 minutes or longer needs to be secured.

(Setting Time)

The setting time was measured for the gypsum compositions for a mold of Examples and Comparative Examples in accordance with JIS T6604 Dental Plaster-5.5 Setting Time Test. Specifically, the measurement was performed through operation according to the procedures described below.
A ring mold for setting time measurement is placed at the center of a glass plate.
Gypsum slurries are obtained by the same method as in measuring the above-described “pot life,” and the gypsum slurries are individually poured into the ring mold for setting time measurement until each gypsum slurry rose a little above the upper face of the ring mold for setting time measurement.
The gypsum slurry is levelled with a spatula to adjust the height of the gypsum slurry to the upper end of the ring mold for setting time measurement.
Using a Vicat apparatus (1 mm needle, 300 g), a needle is made to fall gently under its own weight from the surface of the gypsum slurry 1 minute or 2 minutes before the expected setting time.
The mold for setting time measurement is moved with the glass plate so that the next needle can be made to fall gently under its own weight from the specimen surface to a new part at least 5 mm apart from the wall of the mold and the mark where another needle has been penetrated.
After the needle is wiped cleanly, the tip of the needle is made to be in contact with the surface of the gypsum slurry, and the rod is fixed with a set screw.
The scale is read, the set screw is loosened at 15 second intervals, and the rod is released.
The time required after the powdery gypsum composition for a mold comes into contact with water until the needle penetration depth is 2 mm or less is defined as “setting time.”
The goal of the setting time in the present invention was set to within 30 minutes because long setting time inhibits moving on to the next operation.

(Bleeding of Water During Kneading)

“Bleeding of water during kneading” was checked when the above-described “setting time” was measured because, as described below, the testing operation is partially the same as that in the above-described measurement of setting time, performed for the gypsum compositions for a mold.
A ring mold for setting time measurement is placed at the center of a glass plate.
The gypsum compositions for a mold of Examples and Comparative Examples, which are objects of the measurement, are individually malaxated, and resultant gypsum slurries are individually poured into the ring mold for setting time measurement until each gypsum slurry rose a little above the upper face of the ring mold for setting time measurement.
The gypsum slurry is levelled with a spatula to adjust the height of the gypsum slurry to the upper end of the ring mold for setting time measurement.
Whether or not water bleeds out on the upper face of the ring mold by the time when the needle penetration depth of the Vicat needle apparatus reaches 1 mm from the lower face of the ring mold is visually observed and evaluated.

(Measurement of Flow)

The flow value was measured for the gypsum compositions for a mold of Examples and Comparative Examples in accordance with JIS T6604 Dental Plaster-5.4 Fluidity Test. Specifically, the measurement was performed through operation according to the procedures described below.
A glass plate is placed on a vibration-free surface, and a ring mold is placed at the center of this glass plate.
Each gypsum slurry is poured into the ring mold until it reaches the upper face of the ring.
The ring mold having a cylindrical shape of 50 mm height and 35 mm inner diameter is vertically lifted from the glass plate at a rate of about 10 mm/s 2 minutes and 15 seconds after malaxation is started, which thereby allows the gypsum slurry poured into the ring mold to spread on the glass plate.
The maximum diameter and minimum diameter of the spread of the bottom part of the gypsum slurry are measured in units of mm 1 minute after the ring is lifted, and the arithmetic average value determined from the measured values is defined as the flow value.

(Compressive Strength)

The compressive strength was measured, in accordance with JIS T6604 Dental Plaster-5.8 Compressive Strength Test, for the gypsum set products obtained using the gypsum compositions for a mold, which are objects of the measurement. Specifically, the measurement was performed according to the following procedures.
For the measurement of the compressive strength, a quintet of molds for compressive strength having a cylindrical shape of 40 mm height and 20 mm inner diameter is used.
The molds for compressive strength on which a mold release agent has been applied are placed at the center of the glass plate.
The gypsum compositions for a mold, which are objects of the measurement, are individually malaxated, and resultant gypsum slurries are individually poured into the ring mold for compressive strength on the glass plate until each gypsum slurry rose a little above the edge of the ring mold. On this occasion, the mold is calmly vibrated during pouring the gypsum slurry in order to minimize mixing of bubbles.
Before the malaxated product is set and gloss disappears from the surface of the malaxated product, a glass plate on which a mold release agent has been applied is pressure-bonded to the upper face of the mold.
The sample of the gypsum set product for compressive strength measurement is taken out of the mold 45 minutes after the malaxation is started.
A breaking test is performed on the 5 samples of the gypsum set products for compressive strength measurement using a compressive strength tester 60 minutes after the malaxation is started, and the maximum force (F) applied is recorded.

Then, the compressive strength (S) was calculated according to the following equation.

S=F/314

The goal of the compressive strength in present invention is set to “10 MPa or more” as the strength of a gypsum set product which is a mold obtained using the gypsum composition for a mold for tertiary investing, so that the set product of the gypsum composition, which is used for secondary investing, will not break when the resin for forming artificial gingiva (gum) for dentures made of resin is injected.

TABLE 1
Compositions of gypsum compositions, and properties of kneaded products and gypsum set products
obtained from the gypsum compositions

| | | Com. | | | | | | Com. |
|---|---|---|---|---|---|---|---|---|
| | | Ex. 1 | Ex. 1 | EX. 2 | EX. 3 | EX. 4 | Ex. 5 | Ex. 2 |
| Mixing water amount [%] | | 45 | 45 | 45 | 45 | 45 | 45 | 45 |
| Gypsum | α Gypsum | 20 | 25 | 30 | 40 | 60 | 65 | 70 |
| hemihydrate | β Gypsum | 80 | 75 | 70 | 60 | 40 | 35 | 30 |

| | | | | | | | |
|---|---|---|---|---|---|---|---|
| Average particle size [μm] | 36.2 | 36.3 | 36.5 | 36.7 | 37.2 | 37.3 | 37.4 |
| Water-reducing agent^{*1} (%/st) | 0.25 | 0.18 | 0.10 | 0.08 | 0.02 | 0.00 | 0.00 |
| Pot life [mm:ss] | 14:30 | 09:00 | 10:00 | 11:30 | 10:30 | 15:00 | — |
| Setting time [mm:ss] | 42:45 | 27:00 | 15:30 | 20:00 | 16:00 | 19:30 | — |
| Flow value [mm] | 119 | 118 | 120 | 115 | 121 | 134 | — |
| Compressive strength [MPa] | 9.1 | 10.3 | 11.5 | 12.8 | 12.8 | 12.0 | — |
| Bleeding of water during kneading | Not | Not | Not | Not | Not | Not | Occurs |
| | occur | occur | occur | occur | occur | occur | |

^{*1}Melflux 6681F (polycarboxylate-based)
*2: Ex. and Com. Ex. denote Example and Comparative Example, respectively.

As shown in Table 1, when the gypsum composition of Comparative Example 1, where the amount of α gypsum hemihydrate is 20 parts, was used, the setting time of gypsum took 42 minutes or longer, but in contrast, when the gypsum compositions of Examples were used, it was ascertained that the setting time can be shortened to about 20 minutes. In addition, as can be seen in Comparative Example 2, where the amount of α gypsum hemihydrate is 70 parts, it was ascertained that bleeding of water occurred during the measurement of the setting time and therefore the gypsum composition of Comparative Example 2 is not suitable for applications of preparing gypsum set products. Note that in the case of the gypsum composition of Comparative Example 2, bleeding of water occurred and therefore evaluations other than bleeding of water during kneading were not performed.

### REFERENCE SIGNS LIST

1: Mold-making gypsum composition (for tertiary investing) of the present invention
2: Wax dentures having artificial teeth and wax occlusion rim
2a: Wax occlusion rim
2b: Artificial teeth
3: Working model
4: Gypsum composition for primary investing
5a: Lower-side container of polymerization flask
5b: Upper-side lid of polymerization flask
6: Gypsum composition for secondary investing
7: Hole for filling gypsum composition for tertiary investing
10: Polymerization flask

1. A mold-making gypsum composition comprising gypsum hemihydrate, wherein the gypsum hemihydrate comprises α gypsum hemihydrate and β gypsum hemihydrate in a ratio of 25:75 to 65:35.

2. The mold-making gypsum composition according to claim 1, wherein the ratio of α gypsum hemihydrate to β gypsum hemihydrate is 25:75 to 60:40.

3. The mold-making gypsum composition according to claim 1, wherein the ratio of α gypsum hemihydrate to β gypsum hemihydrate is 30:70 to 60:40.

4. The mold-making gypsum composition according to claim 1, wherein the gypsum hemihydrate accounts for 95 parts by mass or more of 100 parts by mass of the mold-making gypsum composition.

5. The mold-making gypsum composition according to claim 1, further comprising a water-reducing agent, wherein the amount of the water-reducing agent added is 0.02 to 0.2 parts by mass based on 100 parts by mass of the gypsum hemihydrate.

6. The mold-making gypsum composition according to claim 1, wherein a pot life of a slurry-like malaxated product obtained by adding water to the mold-making gypsum composition in such a way that the mixing water amount is 45% and malaxating the water and the mold-making gypsum composition is adjusted to be 8 minutes or longer, and a setting time thereof is adjusted to 30 minutes or shorter.
